(19) United States
(12) Patent Application Publication
(10) Pub. No.: US 2025/0177203 A1
(43) Pub. Date: Jun. 5, 2025

(54) Phacoemulsification Using Image Processing

(71) Applicant: JOHNSON & JOHNSON SURGICAL VISION, INC., Irvine, CA (US)

(72) Inventors: Vadim Gliner, Haifa (IL); Eran Haas, Haifa (IL)

(21) Appl. No.: 18/528,076

(22) Filed: Dec. 4, 2023

(51) Int. Cl. A61F 9/007; A61F 9/008; A61M 1/00; G06T 7/00

(52) U.S. Cl. CPC A61F 9/00745; A61F 9/008; A61M 1/774; G06T 7/0012; A61F 2009/00887; A61M 2210/0612; G06T 2207/30041

(57) ABSTRACT

Phacoemulsification apparatus having a phacoemulsification probe that has a needle with a distal tip configured to be inserted into an eye lens, the needle having a lumen. The probe has an actuator configured to vibrate the needle, an aspiration channel, coupled with the lumen and configured to convey an emulsified particle of the lens as aspiration fluid, and an aspiration sensor, coupled with the aspiration channel and configured to measure a vacuum level within the aspiration channel. A microscope is configured to capture an image of the distal tip and of the emulsified particle. A processor is configured to provide an analysis of the image, and, in response to the analysis, when the emulsified particle is in contact with the distal tip for at least a preset time period while the vacuum level is below a predetermined threshold, energize the actuator with a pulse of energy.

### FIELD OF THE DISCLOSURE

This disclosure relates generally to phacoemulsification, and specifically to controlling activation of ultrasound to achieve a more efficient surgical procedure.

### BACKGROUND

Phacoemulsification is used in cataract surgery as a methodology in which the natural lens of an eye, which has developed a cataract, is emulsified with the tip of an ultrasonic handpiece and is aspirated from the eye. The aspirated matter may be replaced by irrigation of the eye during the phacoemulsification procedure.

### BRIEF DESCRIPTION OF THE DRAWINGS

The present disclosure will be understood from the following detailed description, taken in conjunction with the drawings in which:

FIG. 1 is a pictorial view of a phacoemulsification apparatus being used for a phacoemulsification procedure, according to an example of the present disclosure;

FIG. 2A is a schematic diagram illustrating an image received from a microscope, according to an example of the present disclosure;

FIG. 2B is a schematic diagram illustrating an image received from a microscope, according to an example of the present disclosure;

FIG. 2C is a schematic diagram illustrating an image received from a microscope, according to an example of the present disclosure;

FIG. 2D is a schematic diagram illustrating an image received from a microscope, according to an example of the present disclosure;

FIG. 3 is a flowchart of steps of an algorithm performed to operate the apparatus of FIG. 1, according to an example of the present disclosure; and

FIG. 4A is a schematic diagram illustrating the flowchart of FIG. 3, according to an example of the present disclosure;

FIG. 4B is a schematic diagram illustrating the flowchart of FIG. 3, according to an example of the present disclosure; and

FIG. 4C is a schematic diagram illustrating the flowchart of FIG. 3, according to an example of the present disclosure.

### DESCRIPTION OF EXAMPLES

### Overview

In a patient with a cataract, the natural lens having the cataract may be removed using a phacoemulsification procedure. In the procedure, the distal tip of a hollow needle of a phacoemulsification handpiece is vibrated ultrasonically by a piezoelectric actuator. The vibrating distal tip may be used to divide the lens into smaller pieces, which are then aspirated via an aspiration channel connected to the hollow needle.

During the phacoemulsification procedure it is advantageous to only drive the actuator when the needle distal tip is in contact with the lens, since driving the actuator when there is no contact dissipates unwanted energy in the eye, and the unwanted energy may cause trauma to the eye.

When a lens particle occludes the needle distal tip the vacuum level in the aspiration channel increases (i.e., the pressure level in the channel decreases). A processor registers the vacuum level measured from a sensor in the aspiration channel, and if the measured vacuum level meets and/or exceeds a predetermined threshold, the processor operates the actuator. (If the pressure is high—corresponding to no or little vacuum, indicating no occlusion—the actuator is not driven.)

However, if a particle only partially covers/blocks the needle distal tip, only a partial occlusion occurs. This partial occlusion may not generate a high enough vacuum to cause the actuator to be driven. Also, even though fluid is being aspirated from the eye, the particle may not be aspirated. For example, this may occur with larger gauge needles. To correct this problem an image of the distal tip may be acquired, and an image processor may be used to ascertain that there is a particle close to or at the distal tip for at least a preset time period. If this is the case, and if there is no high vacuum during the time period, the processor pulses the actuator to dislodge or push the particle away. Typically, the aspirated fluid sucks the dislodged particle back toward the needle distal tip allowing the particle to reengage with the needle distal tip with the intent of achieving better occlusion. The better occlusion generates or enables an increase in vacuum that meets and/or exceeds a predetermined vacuum threshold, thereby causing activation of the actuator and enabling the particle to be emulsified and aspirated.

### System Description

In the following description, like elements are identified by the same numeral, and are differentiated, where required, by having a letter attached as a suffix to the numeral.

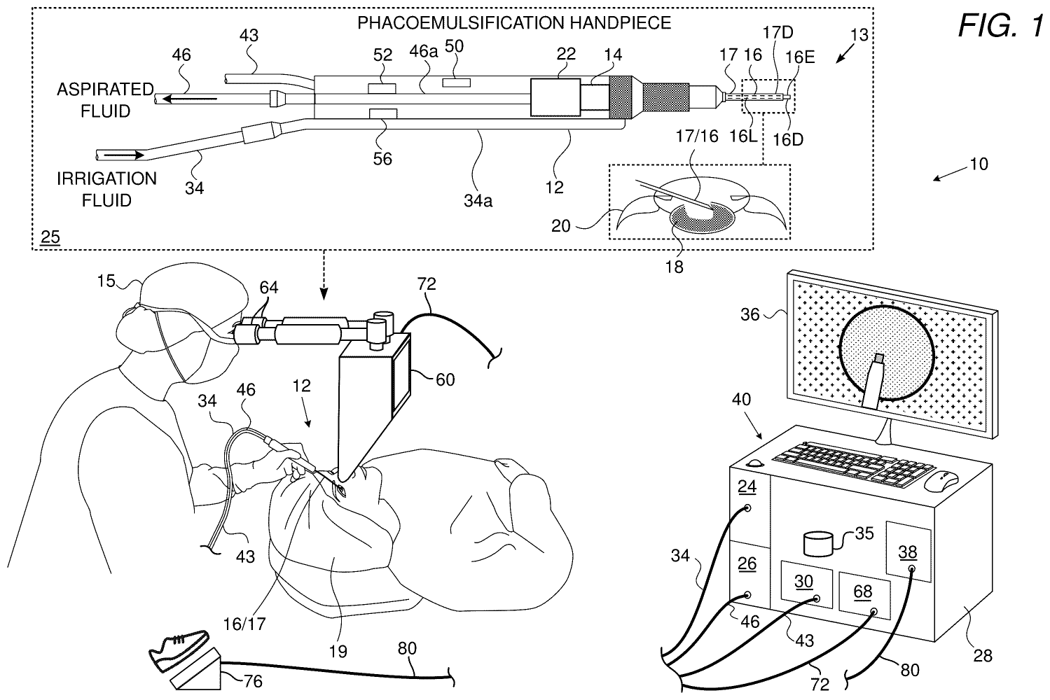

FIG. 1 is a pictorial view of a phacoemulsification apparatus 10 being used for a phacoemulsification procedure, according to an example of the present disclosure. Elements of apparatus 10 are under overall control of a processor 38 in a console 28 and functions of processor 38 are described in more detail below. At least some of the functions of processor 38 may be carried out by suitable software stored in a memory 35. The software may be downloaded to a device in electronic form, over a network, for example. Alternatively, or additionally, the software may be stored in tangible, non-transitory computer-readable storage media, such as optical, magnetic, or electronic memory. Some or all of the functions of processor 38 may be combined in a single physical component or, alternatively, implemented using multiple physical components. The physical components may comprise hard-wired or programmable devices, or a combination of the two.

FIG. 1 includes an inset 25, and as shown in the figure and the inset apparatus 10 includes a phacoemulsification probe/handpiece 12 that comprises a hollow needle 16 and a coaxial irrigation sleeve 17. Irrigation sleeve 17 at least partially surrounds the needle 16 and creates a fluid pathway between the external wall of the needle 16 and the internal wall of the sleeve 17. Needle 16 and sleeve 17 are also herein termed a needle-sleeve combination 13. Needle 16 has a lumen 16L and a distal tip 16D, and sleeve 17 has a distal section 17D.

In the illustrated procedure needle 16 is configured to be inserted into a lens 18 of an eye 20 of a patient 19. Needle 16 is mounted on a horn 14 of probe 12, and is shown in inset 25 as a straight needle. However, any suitable needle may be used with the phacoemulsification probe 12, for example, a curved or bent tip needle that is commercially available from Johnson & Johnson Surgical Vision, Inc., Irvine, CA, USA.

During the phacoemulsification procedure, an irrigation pump 24, which may be in or outside console 28, pumps irrigation fluid through an irrigation channel 34a in handpiece 12 to irrigation sleeve 17 so as to irrigate the eye. The fluid is pumped via an irrigation tubing line 34, running from the pump 24, that is coupled with channel 34a of the probe 12.

An aspiration pump 26, which also may be located in or outside console 28, aspirates aspiration fluid, comprising eye fluid and waste matter (e.g., emulsified parts of the lens and balanced salt solution), from the patient's eye 20 via needle lumen 16L, through an aspiration channel 46a in handpiece 12. Aspiration pump 26 produces a vacuum and is coupled with aspiration channel 46a by an aspiration tubing line 46.

Pumps 24 and 26 may be any pump known in the art (e.g., a peristaltic pump or a progressive cavity pump), and the pumps are both under overall control of processor 38.

A physician 15 holds handpiece 12 so as to perform a phacoemulsification procedure on the eye 20 of patient 19. Handpiece 12 comprises a piezoelectric actuator 22, which, when activated, is configured to vibrate horn 14 and needle 16 in one or more vibration modes of the combined horn and needle. During the phacoemulsification procedure the vibration of needle 16 is used to break up natural lens 18 into small pieces. The physician may set actuator 22 to an operational state wherein the actuator may be driven, i.e., energized, so as to vibrate needle 16. As is described below, the physician may use a foot pedal 76 to set and/or control actuator 22.

In an example of the disclosure, foot-pedal 76 acts as a sub-system user interface, and in a disclosed example the foot-pedal has four positions: a first position where the foot-pedal is not operated such that neither irrigation pump 24 nor aspiration pump 26 are activated, a second position where the irrigation pump alone is activated, a third position where both the irrigation and the aspiration pumps are activated, and a fourth position where actuator 22 is set to its operational state, and in addition the irrigation and aspiration pumps are activated. Foot pedal 76 is connected by a cable 80 to processor 38.

Apparatus 10 comprises an ophthalmic surgical microscope 60, such as ZEISS OPMI LUMERA series or ZEISS ARTEVO series surgical microscopes supplied by Carl Zeiss Meditec AG (Oberkochen, Germany), or any other suitable type of ophthalmic surgical microscope provided by other suppliers. Ophthalmic surgical microscope 60 is configured to acquire and present stereoscopic optical images and two-dimensional (2D) optical images of eye 20. During the phacoemulsification procedure, physician 15 typically looks though eyepieces 64 of ophthalmic surgical microscope 60 for viewing eye 20.

Microscope 60 is also configured to transfer the images it acquires, and that are viewed by physician 15, to an image analyzing module 68 in console 28, and microscope 60 is also herein termed imaging device 60. Imaging device 60 may transfer the images it acquires to module 68 by a cable 72, as illustrated in the figure, and/or may transfer the acquired images wirelessly. In some examples the acquired images may be presented on a display 36. In a disclosed embodiment imaging device 60 generates/captures new images of the scene viewed at the rate of 60 images per second.

Module 68 is operated by processor 38 and is configured to identify the location and orientation of distal tip 16D of needle 16, and of distal section 17D of sleeve 17, in the image received from microscope 60. Module 68, with processor 38, is also configured to identify in the received image the locations of emulsified sections of lens 18.

In order to perform the identifications, module 68 is trained, prior to being used in the procedure referred to herein, with images, similar to those generated/captured by microscope 60, that are recorded in prior cataract surgery operations. The images may be from procedures using apparatus 10, or using apparatus similar to apparatus 10. For simplicity in the following description the training images are assumed to be received from microscope 60 and handpiece 12 operating on eyes other than eye 20, and those having ordinary skill in the art will be able to adapt the description, mutatis mutandis, for surgery images received from other microscopes and other phacoemulsification handpieces.

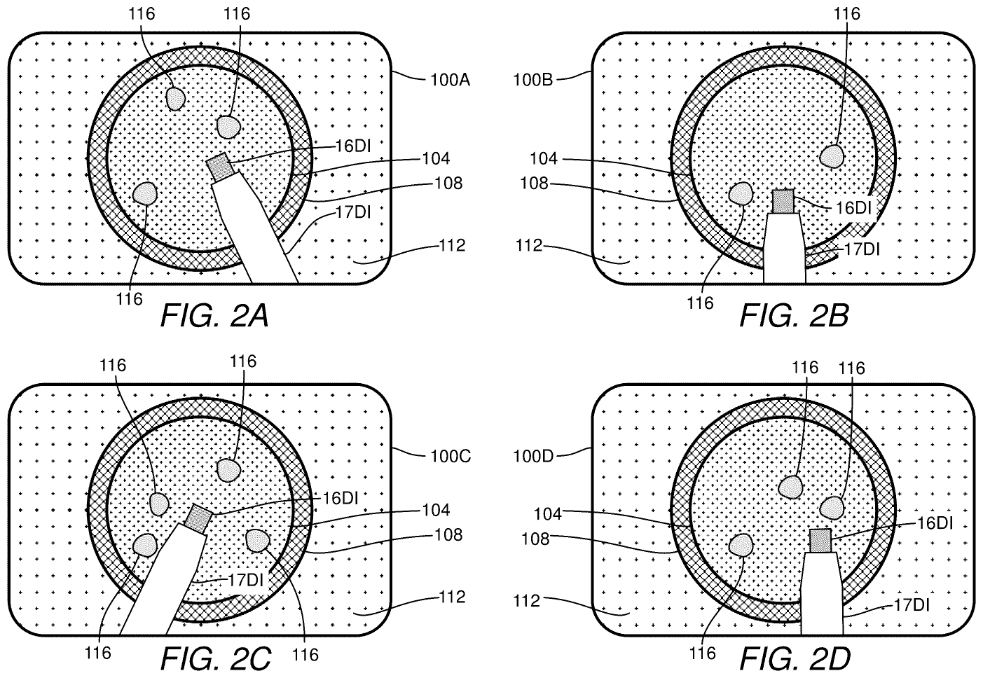

FIGS. 2A, 2B, 2C, and 2D are schematic diagrams illustrating the types of images received from microscope 60 that are used to train module 68, according to an example of the present disclosure. FIGS. 2A, 2B, 2C, and 2D respectively comprise received images 100A, 100B, 100C, and 100D, generically referred to herein as images 100.

In images 100, images of physical elements of apparatus 10 referred to herein are identified by appending the letter “I” to the identifying numeral of the physical element. Thus, images 100, used to train module 68, comprise an image 16DI of needle distal tip 16D, and an image 17DI of distal section 17D of sleeve 17, when needle-sleeve combination 13 is operating on a lens 104 of an eye. In addition, images 100 schematically illustrate a sclera 112 and an iris 108 of the eye, together with emulsified particles 116 of emulsified sections of lens 104.

To train module 68, images 100 are segmented using any convenient segmentation method known in the art. In a disclosed example, color image segmentation is used, typically using images formatted in hue, saturation and value (HSV) parameters. Alternatively, since images 100 comprise images of needle distal tip 16D and sleeve distal section 17D, and since these elements have known dimensions and are in a known, fixed configuration with respect to each other, in an alternative example a model-based segmentation method may be used, typically together with another segmentation method such as an edge-based segmentation method. While emulsified particles 116 do not have known dimensions, in an example they may be considered to be topologically equivalent to a circle, and based on this equivalence a model-based segmentation method together with an edge-based method may also be used for their identification.

Returning to FIG. 1, once module 68 is trained the module may receive an image from microscope 60, and segment the image to identify and find the location and orientation of sleeve distal section 17D and needle distal tip 16D, as well as locations 16E of the edge of the needle distal tip. The module may also segment the image to find locations of emulsified particles of the lens being operated on in the procedure, as well as locations of the edges of the emulsified particles. The segmentation method may be similar to that used for training the module, or alternatively any other suitable segmentation method may be used.

In probe 12, actuator 22 is powered by driving module 30 in console 28. Module 30, under overall control of processor 38, is configured to provide the power to the actuator 22, via e.g., a microcontroller 50 located in the handpiece 12. Power for microcontroller 50, as well as control signals for the microcontroller, is delivered to the microcontroller by a cable 43 from driving module 30.

Processor 38 may receive user-based commands via elements of a system user interface 40, the elements typically comprising a keypad and/or a pointing device. The commands from interface 40 may include, but are not limited to, setting and/or adjusting a vibration mode and/or a frequency of piezoelectric actuator 22, setting and/or adjusting a stroke amplitude of needle 16, and setting and/or adjusting an irrigation flow rate of irrigation pump 24 and an aspiration flow rate and/or a vacuum of aspiration pump 26.

Processor 38 may present setting and parameter information of the phacoemulsification procedure on display 36. In an example, the elements of user interface 40 noted above and display 36 may be one and the same, such as a touch screen graphical user interface.

An irrigation pressure sensor 56 is coupled with irrigation channel 34a, in a section of the channel, so as to couple with the irrigation fluid in the channel. An aspiration pressure sensor 52 is coupled with aspiration channel 46a, in a section of the channel, so as to couple with the aspiration fluid in the channel. In another example, irrigation pressure sensor 56 and aspiration pressure sensor 52 may be coupled anywhere along the irrigation line 34 and aspiration line 46, respectively. The signals generated by the two sensors, sensor 52 and sensor 56, are provided via cable 43 to processor 38.

Physician 15 may use other surgical tools, in addition to probe 12, which are not shown in FIG. 1, to maintain clarity and simplicity in the present description.

As is described in more detail below, in a disclosed example of the present disclosure, processor 38 uses the signals from aspiration pressure sensor 52 to decide if actuator 22 is to be driven, i.e., energized, so as to vibrate needle 16. In the disclosed example, foot pedal 76 is placed in its fourth position, so that processor 38 activates irrigation pump 24 and aspiration pump 26, and places actuator 22 in an operational state. However, processor 38 only energizes the actuator while it is in the operational state if the signals from aspiration pressure sensor 52 indicate there is an occlusion, e.g., the vacuum level meets and/or exceeds a predetermined threshold. In a disclosed example, the predetermined threshold, which may be set by physician 15, may be between approximately 300 mmHg and approximately 700 mmHg.

Presence of a high vacuum that meets and/or exceeds a predetermined threshold indicates that needle distal tip 16D is occluded, and so is in contact with lens 18 or emulsified particles of the lens. In this situation, energy from activated actuator 22 vibrates needle 16 so as to further emulsify lens 18 or the particles 116. If there is no high vacuum such that the predetermine threshold is not met and/or exceeded, needle distal tip 16D is typically not in contact with lens 18 or emulsified particles 116, and in this situation actuator 22 is not activated since any energy from its activation, so as to vibrate needle 16, is unnecessary and could damage eye 20.

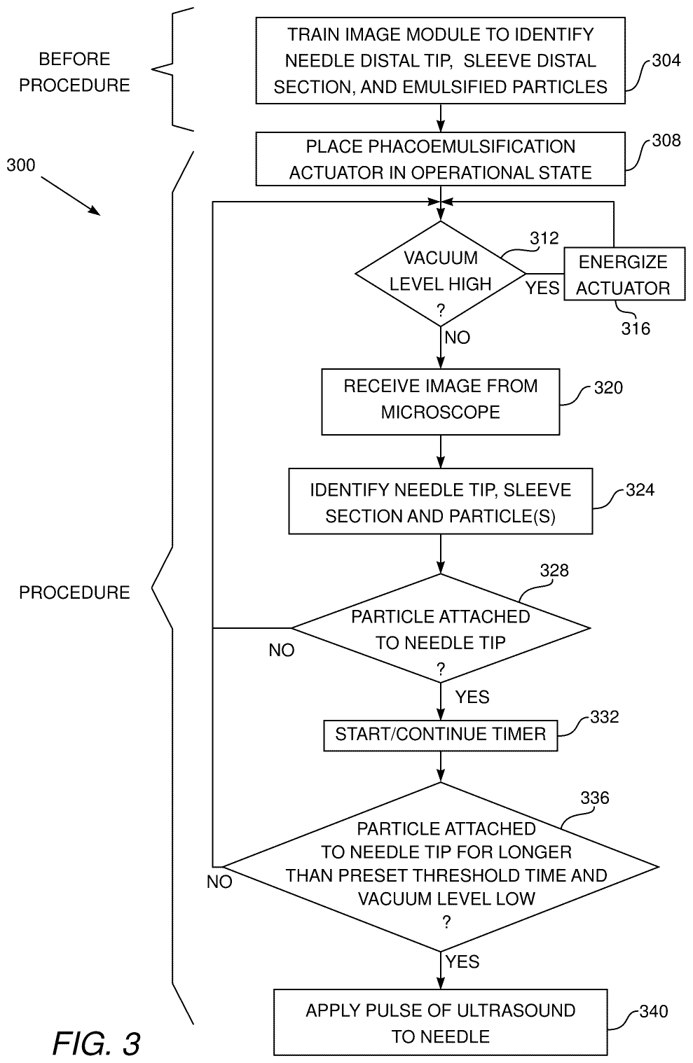

However, the inventors have found there are some situations wherein, while needle tip 16D does contact a lens or an emulsified particle, insufficient vacuum is generated (a threshold is not met and/or exceeded) so that actuator 22 is not energized. The flowchart of FIG. 3, below, describes steps of an algorithm operated by processor 38 to overcome this problem.

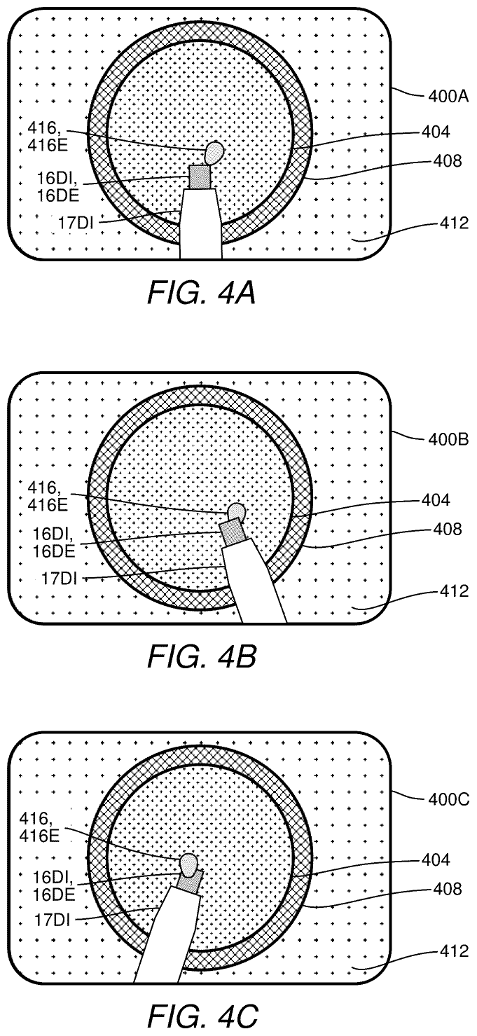

FIG. 3 is a flowchart 300 of steps of an algorithm performed by processor 38 to operate apparatus 10, and FIGS. 4A, 4B, and 4C are schematic diagrams illustrating the flowchart, according to an example of the present disclosure.

In an initial step 304, module 68 is trained. The method of training is substantially similar to that described above using images 100 of FIGS. 2A-2D, so that after training the module it is able to identify and find the location and orientation of needle distal tip 16D, and of a sleeve distal section 17D, in an image received from microscope 60. In addition, the training enables module 68 to find the location, in the received image, of emulsified particles of a lens.

Processor 38 typically performs step 304 prior to the procedure on eye 20 illustrated in FIG. 1. The processor typically performs the remaining steps of the flowchart during the procedure.

In an operational step 308, physician 15 inserts needle-sleeve combination 13 into lens 18. Physician 15 then uses foot-pedal 76 to set actuator 22 to its operational state, by moving the foot-pedal to its fourth position. Thus, irrigation pump 24 and aspiration pump 26 are also activated, in addition to actuator 22 being set to its operational state.

In the operational state, processor 38 may energize actuator 22 to vibrate needle 16 or may leave the actuator unenergized.

In a first decision step 312, processor 38 accesses aspiration pressure sensor 52 to determine if there is a high vacuum level in aspiration channel 46a that meets and/or exceeds a predetermined threshold, indicating that needle lumen 16L is obstructed/occluded. A high vacuum level may be indicated by comparing the vacuum measured from the aspiration pressure sensor 52 with a vacuum threshold set by the user or provided as a default setting of the system. If the measured vacuum level meets and/or exceeds the predetermined threshold then a high vacuum level is determined. If there is a high vacuum level, i.e., if decision step 312 returns positive, then in an energization step 316 processor 38 energizes actuator 22 to vibrate needle 16, and the flowchart returns reiteratively to the decision step.

If decision step 312 returns negative, i.e., if there is a low vacuum level in aspiration channel 46a (the vacuum level does not meet and/or exceed the set/predetermined threshold), then processor 38 does not energize actuator 22, and the flowchart proceeds to an imaging step 320.

In imaging step 320, processor 38 accesses an image of eye 20 generated/captured by microscope 60. FIGS. 4A, 4B, and 4C, respectively schematically illustrate exemplary images 400A, 400B, and 400C, generically termed images 400, that are accessed. Images 400 exemplify the changing nature of the scene imaged by microscope 60, as well as different possible scenarios of the scene. The different scenarios are described below.

Images 400 show images of a sclera 412, an iris 408, and a lens 404 of eye 20. An image 416 of an emulsified particle of the eye lens, image 16DI of the distal tip 16D, and image 17DI of the distal section 17D of sleeve 17 are also shown.

In an identification step 324, the processor uses module 68 to identify the location and orientation of image 16DI of needle distal tip 16D, as well as locations 16DE of the edges of tip 16D. The processor may use the known configuration of the needle distal tip and the distal sleeve section to aid in the identification of image 16DI.

Also in step 324, processor 38 uses module 68 to identify locations of emulsified particles 416 of lens 18 that are adjacent to and contact or even partially cover distal tip 16D. By way of example emulsified particle 416 is illustrated in images 400. For each emulsified particle identified, processor 38 finds the locations of the edges of the particle. Thus, for particle 416, processor 38 determines locations 416E of the edges of the particle.

In a second decision step 328, processor 38 checks if emulsified particle 416 is in contact with needle distal tip 16D. There are several possible scenarios of contact, including:

Particle 416 butts distal tip 16D, as illustrated in image 400A;

Distal tip 16D obscures a part of particle 416, as illustrated in image 400B; or

A part of particle 416 obscures distal tip 16D, as illustrated in image 400C.

The two-dimensional (2D) images received from microscope 60 do not differentiate the first two scenarios. Thus, butting attachment or attachment where tip 16D obscures part of particle 416 can be ascertained if there are at least some edges 416E and 16DE that have common locations.

The third scenario can be ascertained if locations of some edges 416E of particle 416 lie within distal tip 16D, i.e., if some of edges 416E overlay distal tip image 16DI.

If, in the image being checked, any of the above scenarios is valid, then decision step 328 returns positive, i.e., yes, and the flowchart continues to a timing step 332. If none of the scenarios is valid, then decision step 328 returns negative, i.e., no, and the flowchart returns to first decision step 312.

It will be understood that when decision step 312 and decision step 328 both return negative, i.e., when the vacuum level is low (does not meet and/or exceed the predetermined threshold) and there is no particle in contact with the needle distal tip, steps 312-328 reiterate, and at each iteration, in imaging step 320 a new image is received from microscope 60.

When decision step 312 returns negative and decision step 328 returns positive, there is also reiteration, of steps 312-328 as well as a timing step 332 and a third decision step 336, described below. Also in this case, in each iteration in imaging step 320 a new image is received from microscope 60.

In timing step 332, if this is the first time the step is accessed, processor 38 initiates a timer. If the step has previously been accessed, then processor 38 updates the timer.

The flowchart then continues to third decision step 336. In step 336, processor 38 checks if, while the vacuum level is low, a particle such as particle 416 is attached, as measured in step 328, to distal tip 16D for a time longer than a preset threshold time. The inventors have found that hard particles may take approximately 1 second to emulsify, so that in a disclosed example, the preset threshold time is between approximately 2 seconds and approximately 3 seconds. However, other examples may have shorter or longer preset threshold times.

If decision step 336 returns negative, i.e., no, then the flowchart returns to first decision step 312, so that steps 312-336 may reiterate.

If the decision returns positive, i.e., yes, then in an energizing step 340, processor 38 applies a pulse of ultrasound energy to needle 16, by activating for a short time actuator 22. In a disclosed example the pulse is approximately 100 ms long, but other examples may have shorter or longer pulse times. The inventors have found that applying such a pulse frees a particle that is attached to the needle distal tip, so that the particle may be aspirated from the patient's eye via lumen 16L and aspiration channel 46a.

### EXAMPLES

Example 1. Phacoemulsification apparatus (10), comprising:
a phacoemulsification probe (12), comprising:
a needle (16) having a distal tip (16D) configured to be inserted into a lens (18) of an eye (20), the needle comprising a lumen (16L);
an actuator (22), coupled with the needle, configured when energized to vibrate the needle;
an aspiration channel (46a), coupled with the lumen and configured to convey an emulsified particle (416) of the lens as aspiration fluid; and
an aspiration sensor (52), coupled with the aspiration channel and configured to measure a vacuum level within the aspiration channel;
a microscope (60), configured to capture an image of the distal tip and of the emulsified particle; and
a processor (38), configured to provide an analysis of the image, and, in response to the analysis, when the emulsified particle is in contact with the distal tip for at least a preset time period while the vacuum level is below a predetermined threshold, energize the actuator with a pulse of energy.

Example 2. The phacoemulsification apparatus of example 1, wherein the analysis comprises the processor segmenting the image to identify the distal tip and the emulsified particle, and determining contact in response to the segmenting.

Example 3. The phacoemulsification apparatus of any of examples 1-2, wherein the probe comprises a sleeve, at least partially surrounding the needle and being in a preset configuration with respect to the needle, and wherein the processor is configured to identify the distal tip of the needle in response to the preset configuration.

Example 4. The phacoemulsification apparatus of any of examples 1-3, wherein the analysis comprises the processor identifying tip edge locations of the distal tip and particle edge locations of the emulsified particle, and determining contact if at least some of the tip edge locations and emulsified particle edge locations are common.

Example 5. The phacoemulsification apparatus of any of examples 1-4, wherein the analysis comprises the processor identifying particle edge locations of the emulsified particle, and a location of the distal tip, and determining contact if at least some of the particle edge locations overlay the distal tip.

Example 6. The phacoemulsification apparatus of any of examples 1-5, wherein the preset time period is between 2 seconds and 3 seconds.

Example 7. The phacoemulsification apparatus of any of examples 1-6, wherein the pulse of energy has a duration of at least 100 ms.

Example 8. The phacoemulsification apparatus of any of examples 1-7, wherein the predetermined threshold is in a range from 300 mmHg to 700 mmHg.

Example 9. A method, comprising:
providing a phacoemulsification probe (12), comprising:
a needle (16) having a distal tip (16D) configured to be inserted into a lens (18) of an eye (20), the needle comprising a lumen (16L);
an actuator (22) coupled with the needle and configured when energized to vibrate the needle;
an aspiration channel (46a) coupled with the lumen and configured to convey an emulsified particle (416) of the lens as aspiration fluid; and
an aspiration sensor (52) coupled with the aspiration channel and configured to measure a vacuum level within the aspiration channel;
capturing an image of the distal tip and of the emulsified particle; and
analyzing the image, and, in response to the analyzing, when the emulsified particle is in contact with the distal tip for at least a preset time period while the vacuum level is below a predetermined threshold, energizing the actuator with a pulse of energy.

Example 10. The method of example 9, wherein the analyzing comprises segmenting the image to identify the distal tip and the emulsified particle, and determining contact in response to the segmenting.

Example 11. The method of any of examples 9-10, wherein the probe comprises a sleeve, at least partially surrounding the needle and being in a preset configuration with respect to the needle, and identifying the distal tip in response to the preset configuration.

12. The method of any of examples 9-11, wherein the analyzing comprises identifying tip edge locations of the distal tip and particle edge locations of the emulsified particle, and determining contact if at least some of the tip edge locations and particle edge locations are common.

13. The method of any of examples 9-12, wherein the analyzing comprises identifying particle edge locations of the emulsified particle, and a location of the distal tip, and determining contact if at least some of the particle edge locations overlay the distal tip.

14. The method of any of examples 9-13, wherein the preset time period is between 2 seconds and 3 seconds.

15. The method of any of examples 9-14, wherein the pulse of energy has a duration of at least 100 ms.

16. The method of any of examples 9-15, wherein the predetermined threshold is in a range from 300 mmHg to 700 mmHg.

The examples described above are cited by way of example, and the present disclosure is not limited by what has been particularly shown and described hereinabove. Rather the scope of the disclosure includes both combinations and sub-combinations of the various features described hereinabove, as well as variations and modifications thereof which would occur to persons skilled in the art upon reading the foregoing description and which are not disclosed in the prior art.

1. Phacoemulsification apparatus, comprising:
a phacoemulsification probe, comprising:
a needle having a distal tip configured to be inserted into a lens of an eye, the needle comprising a lumen;
an actuator, coupled with the needle, configured when energized to vibrate the needle;
an aspiration channel, coupled with the lumen and configured to convey an emulsified particle of the lens as aspiration fluid; and
an aspiration sensor, coupled with the aspiration channel and configured to measure a vacuum level within the aspiration channel;
a microscope, configured to capture an image of the distal tip and of the emulsified particle; and
a processor, configured to provide an analysis of the image, and, in response to the analysis, when the emulsified particle is in contact with the distal tip for at least a preset time period while the vacuum level is below a predetermined threshold, energize the actuator with a pulse of energy.

2. The phacoemulsification apparatus of claim 1, wherein the analysis comprises the processor segmenting the image to identify the distal tip and the emulsified particle, and determining contact in response to the segmenting.

3. The phacoemulsification apparatus of claim 2, wherein the probe comprises a sleeve, at least partially surrounding the needle and being in a preset configuration with respect to the needle, and wherein the processor is configured to identify the distal tip of the needle in response to the preset configuration.

4. The phacoemulsification apparatus of claim 1, wherein the analysis comprises the processor identifying tip edge locations of the distal tip and particle edge locations of the emulsified particle, and determining contact if at least some of the tip edge locations and emulsified particle edge locations are common.

5. The phacoemulsification apparatus of claim 1, wherein the analysis comprises the processor identifying particle edge locations of the emulsified particle, and a location of the distal tip, and determining contact if at least some of the particle edge locations overlay the distal tip.

6. The phacoemulsification apparatus of claim 1, wherein the preset time period is between 2 seconds and 3 seconds.

7. The phacoemulsification apparatus of claim 1, wherein the pulse of energy has a duration of at least 100 ms.

8. The phacoemulsification apparatus of claim 1, wherein the predetermined threshold is in a range from 300 mmHg to 700 mmHg.

9. A method, comprising:
providing a phacoemulsification probe, comprising:
a needle having a distal tip configured to be inserted into a lens of an eye, the needle comprising a lumen;
an actuator coupled with the needle and configured when energized to vibrate the needle;
an aspiration channel coupled with the lumen and configured to convey an emulsified particle of the lens as aspiration fluid; and
an aspiration sensor coupled with the aspiration channel and configured to measure a vacuum level within the aspiration channel;
capturing an image of the distal tip and of the emulsified particle; and
analyzing the image, and, in response to the analyzing, when the emulsified particle is in contact with the distal tip for at least a preset time period while the vacuum level is below a predetermined threshold, energizing the actuator with a pulse of energy.

10. The method of claim 9, wherein the analyzing comprises segmenting the image to identify the distal tip and the emulsified particle, and determining contact in response to the segmenting.

11. The method of claim 10, wherein the probe comprises a sleeve, at least partially surrounding the needle and being in a preset configuration with respect to the needle, and identifying the distal tip in response to the preset configuration.

12. The method of claim 9, wherein the analyzing comprises identifying tip edge locations of the distal tip and particle edge locations of the emulsified particle, and determining contact if at least some of the tip edge locations and particle edge locations are common.

13. The method of claim 9, wherein the analyzing comprises identifying particle edge locations of the emulsified particle, and a location of the distal tip, and determining contact if at least some of the particle edge locations overlay the distal tip.

14. The method of claim 9, wherein the preset time period is between 2 seconds and 3 seconds.

15. The method of claim 9, wherein the pulse of energy has a duration of at least 100 ms.

16. The method of claim 9, wherein the predetermined threshold is in a range from 300 mmHg to 700 mmHg.
